(19) United States
(12) Patent Application Publication
(10) Pub. No.: US 2019/0254405 A1
(43) Pub. Date: Aug. 22, 2019

(54) Cosmetic Pad and Method for Producing a Cosmetic Pad

(71) Applicant: Kai-Wing MAN-NEGASI, Mannheim (DE)

(72) Inventor: Kai-Wing MAN-NEGASI, Mannheim (DE)

(21) Appl. No.: 16/347,226

(22) PCT Filed: Nov. 3, 2017
(86) PCT No.: PCT/EP2017/001276

§ 371 Date: May 3, 2019

(30) Foreign Application Priority Data
Nov. 3, 2016 (DE) 10 2016 013 067.9

(51) Int. Cl. A45D 29/00; A61K 8/02; A61Q 3/04; D04H 1/40

(52) U.S. Cl. CPC A45D 29/007; A61K 8/0208; A45D 2200/1063; D04H 1/40; A45D 2200/1018; A61Q 3/04

(57) ABSTRACT

The invention concerns a cosmetic pad for removing nail varnish remover or make-up remover, characterised in that it has an absorbent core and a fleece layer, as well as a method for producing a cosmetic pad.

### BACKGROUND OF THE INVENTION

Cotton wool pads are often used for cosmetic applications, for example for removing make-up. Cotton wool pads normally consist of fibres which are held together in a loose structure based on adhesion of the fibres to each other. It is known to use cotton fibres for cotton wool pads.

### SUMMARY OF THE INVENTION

It is the task of the present invention to provide an improved cotton wool pad, so that the removal of cosmetic residue on skin or nails is simplified.

The solution of the task according to the invention is realised with the features of the independent claims. Advantageous further developments of the invention result from the dependent claims.

According to the invention a cosmetic pad for removal of nail varnish remover or make-up remover is provided, wherein the cosmetic pad has an absorbent core and a fleece layer. The fleece layer serves for absorbing and storing fluid. It for example absorbs fluids or liquid cosmetic residues on the skin or nails and stores the same. This also applies for soluble cosmetic residues which may still adhere to the fleece. The fleece layer has the function of an absorber layer of the cosmetic pad. It can be an absorber layer. The absorbent core stores fluids or liquid cosmetic residues. The absorbent core also serves for an appropriate dispensation of the fluid or liquid cosmetic residues. In some embodiment examples the absorbent core has cellulose flakes and a superabsorber granulate. A superabsorber granulate, for example superabsorbent polymers (SAPs), is capable of absorbing a multiple of fluid, for example water or aqueous solutions.

The cosmetic pad according to the invention is perforated in a further development, so that it can be divided into two parts. The fleece layer can have an embossing such as for example a flower pattern or nubs for this, which are embossed into the fleece material. In some of the following further developments and embodiment examples this embossing also effects an adhesion of the cellular flakes to a fluid-impermeable layer, so that the cellular flakes cannot distribute loosely between the fleece layer and the fluid-impermeable layer. Furthermore, the storage of fluids can be improved in the areas where the fleece layer has been embossed, as the fibres of the layers lie closer together thanks to the embossing in this area and support the storage of fluids. The fleece layer(s) can have surface structures that differ from each other. A rougher structure will for example improve the abrasion and removal of rougher dirt on the skin or nails, wherein a finer structure is more suitable for post-polishing.

A further development of the cosmetic pad envisages that the pad has a distribution covering between the absorbent core and the fleece layer. The distribution covering serves for a faster conduction of fluid or liquid cosmetic residues to the absorbent core. The absorbent core stores the fluid or liquid cosmetic residues. It also serves for an adequate dispensing of the fluid or liquid cosmetic residues back into the fleece layer.

According to another further development of the cosmetic pad it is envisaged that the pad has a gluing between the fleece layer and the distribution covering. The gluing enables an improved connection between the fleece layer and the distribution covering. The gluing is for example realised by means of one, or a combination of adhesives.

In a further development of the cosmetic pad it is envisaged that the pad has a fluid-impermeable layer, in particular a film or a paper, an absorbent core and a fleece layer. The fluid-impermeable layer can be designed as a protective film. The fluid-impermeable layer supports the storage and dispensing of fluid in the absorbent core. The fluid-impermeable layer, in particular the film or the paper, enables double-sided uses of the pad that are separate from each other.

According to another further development of the cosmetic pad it is envisaged that a further absorbent core and a further fleece layer are arranged on the exposed side of the fluid-impermeable layer as well. In order to achieve an even better removal result in some embodiment examples the fluid-impermeable layer separates two absorbent core layers of the absorbent core in order to be able to ensure optimal double-sided use. In order words, the fluid-impermeable layer is arranged in an internal, middle area of the absorbent core.

In one further development of the cosmetic pad it envisaged that the absorbent core and the various layers are connected in the edge area. The fleece layer and the absorbent core are compressed under pressure in an edge area. This compression creates a thin layer. The edge area or edge forms a closed surface. The edge ensures an adhesion of the various layers for maintaining the special absorption capacity inside the pad and for avoiding a separation of the layers through rubbing on a surface. The edge also additionally serves for realising a lint-free result. Compression is realised as a mixture of gluing and embossing if the fleece layer has plastic components, so that the fleece layer(s) and the absorbent core fuse with each other at least in part in the edge area through hot-pressing one on top of the other.

According to another further development of the cosmetic pad it is envisaged that the pad is saturated with a fluid, in particular nail varnish remover or make-up remover. The excellent absorption and storage of fluid enables the cosmetic pad in particular to store the nail varnish remover or make-up remover in an excellent way. The absorbent core in particular allows not only a storage, but also a simultaneous dispensing of the fluid through the fleece layer onto the skin, nails or toe nails. Dispensing the fluid can for example be realised by pressing the pad.

According to a further development of the cosmetic pad it is envisaged that the pad has a round, oval or rectangular shape. Such a shape of the pad supports the handling and the application of fluids or the collection of fluids or cosmetic residues. With a rectangular shape the pad has a length and a width of approx. 5.5 or approx. 4.5 cm with a tolerance of 0.2-0.5 cm each. With an oval shape of the pad the length is approx. 9.0 cm and the width approx. 7.2 cm with a tolerance of 0.2-0.5 cm each.

In a further development of the cosmetic pad it is envisaged that the pad has a diameter, a length or a width of 2-10 cm, in particular of 3-6 cm, in particular of 5.5 or 4.5 cm. The size information includes a tolerance of 0.2-0.5 cm each.

In another further development of the cosmetic pad it is envisaged that the pad has a thickness of 3.0 mm to 6.0 mm, in particular of 3.5 to 5.0 mm, or of 3.0 mm to 4.5 mm. The size information includes a tolerance of approximately 0.5-1.0 mm each.

According to the invention a method for producing, in particular a said cosmetic pad, is also provided.

Advantageous further developments of the invention result from the dependent claims.

### BRIEF DESCRIPTION OF THE DRAWINGS

Further details of the invention can be found in the embodiment examples, which are described below with reference to the Figures. Shown are:

FIG. 1: an embodiment example of a cosmetic pad,

FIG. 2: the cosmetic pad from FIG. 1 in a perspective view,

FIG. 3: a further embodiment example of a cosmetic pad,

FIG. 4: the cosmetic pad from FIG. 3 in a perspective view,

FIG. 5: a further embodiment example of a cosmetic pad,

FIG. 6: the cosmetic pad from FIG. 5 in a perspective view,

FIG. 7: a further embodiment example of a cosmetic pad,

FIG. 8: a further embodiment example of a cosmetic pad,

FIG. 9: two embodiment examples of an edge area of a cosmetic pad.

### DETAILED DESCRIPTION OF THE FIGURES

Examples of embodiments of the invention will be described below with reference to the enclosed drawings:

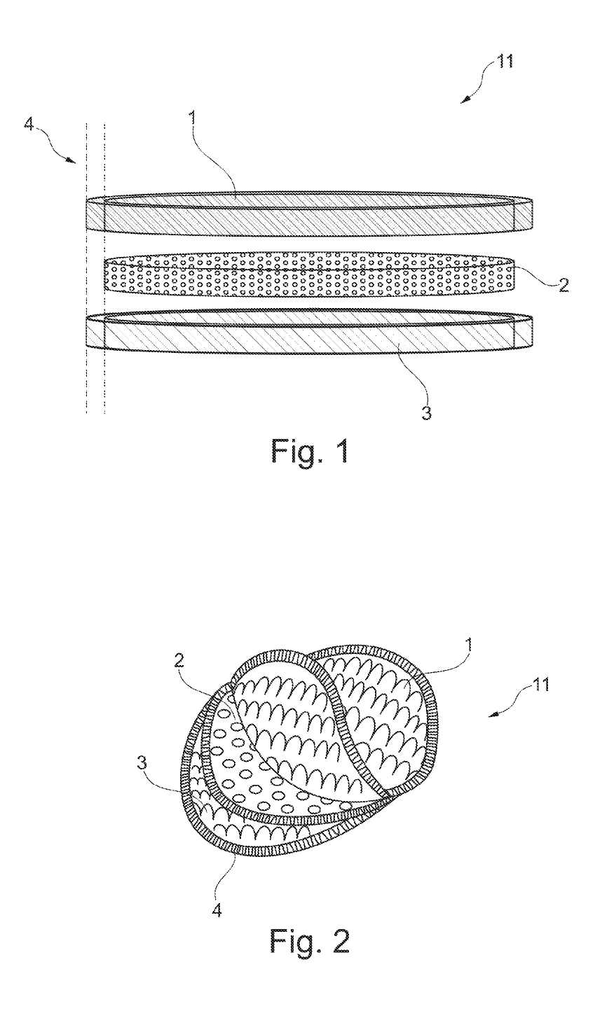

FIG. 1 shows an embodiment example of a cosmetic pad 11. Individual layers of the cosmetic pad 11 are shown at a distance from each other to obtain a better overview.

The upper material of the cosmetic pad 11 has a fleece layer 1, which has the function of collecting and storing fluid. The fleece layer 1 borders on an absorbent core 2, which stores the fluid. The absorbent core also serves for an appropriate dispensing of the fluid back into fleece layer 1 and into fleece layer 3. A cosmetic pad 11 with an upper fleece layer 1 and a lower fleece layer 3 is shown in FIG. 1. There are however embodiment examples in which the cosmetic pad 11 has only one fleece layer 1, 3 and one absorbent core 2.

The fleece layers 1, 3 are firmly compressed under pressure in the edge area 4, so that the edge area 4 is formed as a thin layer and forms a closed surface. The edge 4 ensures an adhesion of the various layers 1, 2, 3 to maintain the special absorption capacity inside the pad and to avoid a separation of the layers 1, 2, 3, for example through rubbing on a surface. The edge 4 also serves for realising an absolutely lint-free result.

In one embodiment example, in which one or more fleece layers 1, 3 include plastic components, compression takes place as a mixture of gluing and embossing, so that the fleece layer(s) 1, 3 and the absorbent core 2 fuse with each other at least in part in the edge area 4 through hot-pressing one on top of the other.

The cosmetic pad has a diameter of approx. 5.5 cm and a thickness of approx. 3.0 mm to 4.5 mm.

FIG. 2 shows a perspective view of the cosmetic pad 11 from FIG. 1. Individual fleece layers 1, 3 as well as the absorbent core 2 are shown spread out on one side for improved understanding. Also easy to see is the edge area 4 of the individual layers of the cosmetic pad 11.

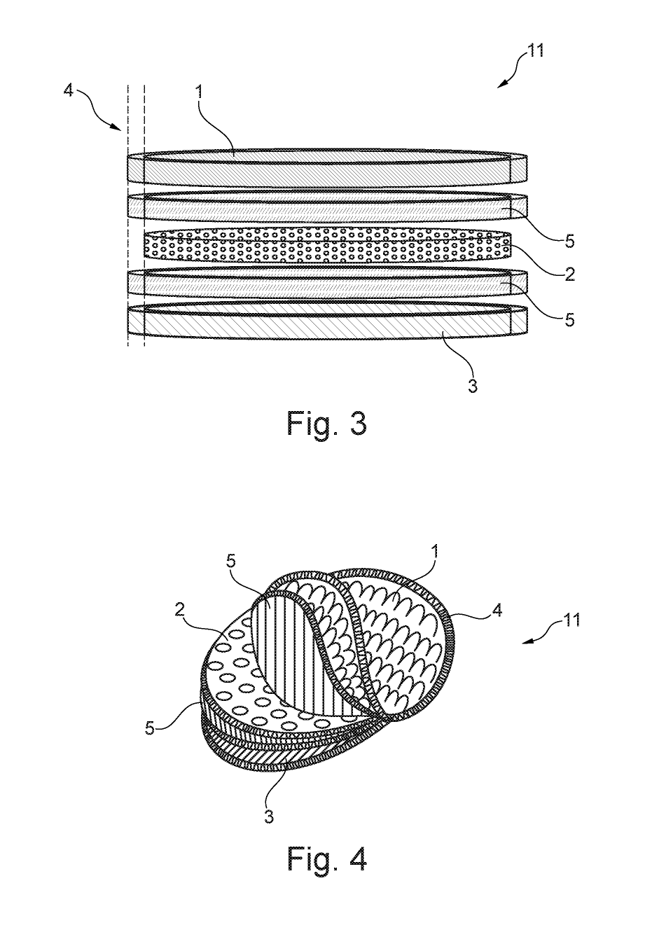

FIG. 3 shows a further embodiment example of a cosmetic pad 11. Individual layers of the cosmetic pad 11 are shown at a distance from each other to obtain a better overview.

The upper material of the cosmetic pad 11 has a fleece layer 1 and has the function of collecting and storing fluids. The fleece layer 1 borders on a distribution covering 5, which ensures a rapid forwarding of fluid to the absorbent core 2. The absorbent core 2 stores the fluid, whilst also serving for an appropriate dispensing of the fluid back into the upper/lower distribution covering 5 and the upper/lower fleece layer 1, 3. There are also embodiment examples where the cosmetic pad 11 has only one fleece layer 1, 3, one distribution covering 5 and one absorbent core 2.

The fleece layers 1, 3 and the distribution covering 5 (or the distribution layer) are firmly compressed under pressure in the edge area 4, so that a thin layer is created and the edge 4 forms a closed surface. The edge 4 ensures an adhesion of the various layers 1, 2, 3, 5 in order to maintain the special absorption capacity inside the pad and for avoiding a separation of the layers 1, 2, 3, 5, for example through rubbing on a surface. The edge 4 also additionally serves for realising an absolutely lint-free result.

The cosmetic pad has a diameter of approx. 5.5 cm and a thickness of approx. 4.0 mm to 5.0 mm.

In an embodiment example, in which one or more fleece layers 1, 3 include plastic components, compression is realised as a mixture of gluing and embossing, so that the fleece layer(s) 1, 3 and the absorbent core 2 fuse with each other at least in part in the edge area 4 through hot-pressing one on top of the other.

FIG. 4 shows a perspective view of the cosmetic pad 11 from FIG. 3. Individual fleece layers 1, 3, the distribution covering 5 and the absorption core 2 are shown spread out on one side for improved understanding. Also easy to see is the edge area 4 of the individual layers of the cosmetic pad 11.

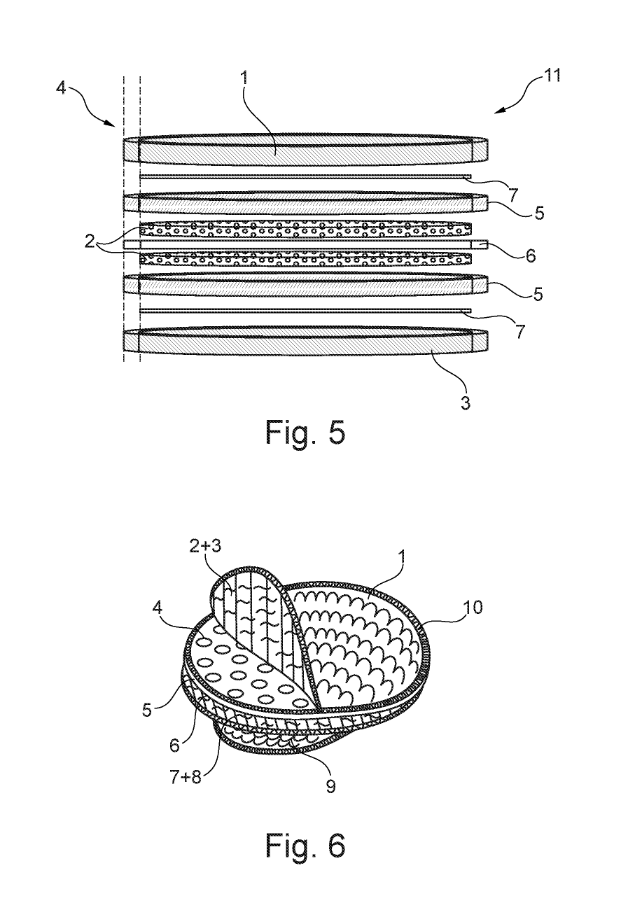

FIG. 5 shows a further embodiment example of a cosmetic pad 11. Individual layers of the cosmetic pad 11 are shown at a distance from each other to obtain a better overview.

The upper material of the cosmetic pad 11 has a fleece layer 1 and has the function of collecting and storing fluid. The fleece layer 1, also called absorber layer, borders on a gluing 7. This is followed by a distribution covering 5, which serves for the rapid forwarding of the fluid to the absorbent core 2. The absorbent core 2 stores the fluid, whilst also serving for an appropriate dispensing of the fluid back into the upper/lower distribution covering 5 and the upper/lower fleece layer 1, 3. The absorbent core 2 contains cellulose and a supergranulate. There are also embodiment examples where the cosmetic pad 11 has only one fleece layer 1, 3, one gluing 7, one distribution covering 5 and one absorbent core 2.

To realise a better removal result a fluid-impermeable layer 6 separates two layers of the absorbent core 2 from each other in order to be able to ensure optimal double-sided use. The fluid-impermeable layer 6 can be arranged centrally between the two layers of the absorbent core 2. The fluid-impermeable layer 6 is a film in this embodiment example, but can also be a paper.

The fleece layers 1, 3, the distribution covering 5 and the fluid-impermeable layer 6 are firmly compressed under pressure in the edge area 4, so that a thin layer is created and the edge 4 forms a closed surface. The edge 4 ensure an adhesion of the various layers 1, 2, 3, 5, 6 in order to maintain the particular absorption capacity inside the pad and for avoiding a separation of the layers 1, 2, 3, 5, 6, for example through rubbing on a surface. The edge 4 also additionally serves for realising an absolutely lint-free result.

The cosmetic pad has a diameter of approx. 5.5 cm and a thickness of approx. 5.0 mm to 6.0 mm.

In one embodiment example, in which the one or more fleece layers 1, 3 include plastic components, compression is realised as a mixture of gluing and embossing, so that the fleece layer(s) 1, 3, the distribution covering 5, the fluid-impermeable layer 6 and the absorbent core 2 fuse with each other at least in part in the edge area 4 through hot-pressing one on top of the other. The fluid-impermeable layer 6, which can be designed as a film, includes plastic components in some embodiment examples, so that their gluing, embossing can be improved by the hot-pressing of the layers on top of the other.

FIG. 6 shows a perspective view of the cosmetic pad 11 from FIG. 5. Individual fleece layers 1, 3, the distribution covering 5 with the gluing 7, the fluid-impermeable layer 6, and the two-part absorbent core 2 are shown spread out on one side for improved understanding. Also easy to see is the edge area 4 of the individual layers of the cosmetic pad 11.

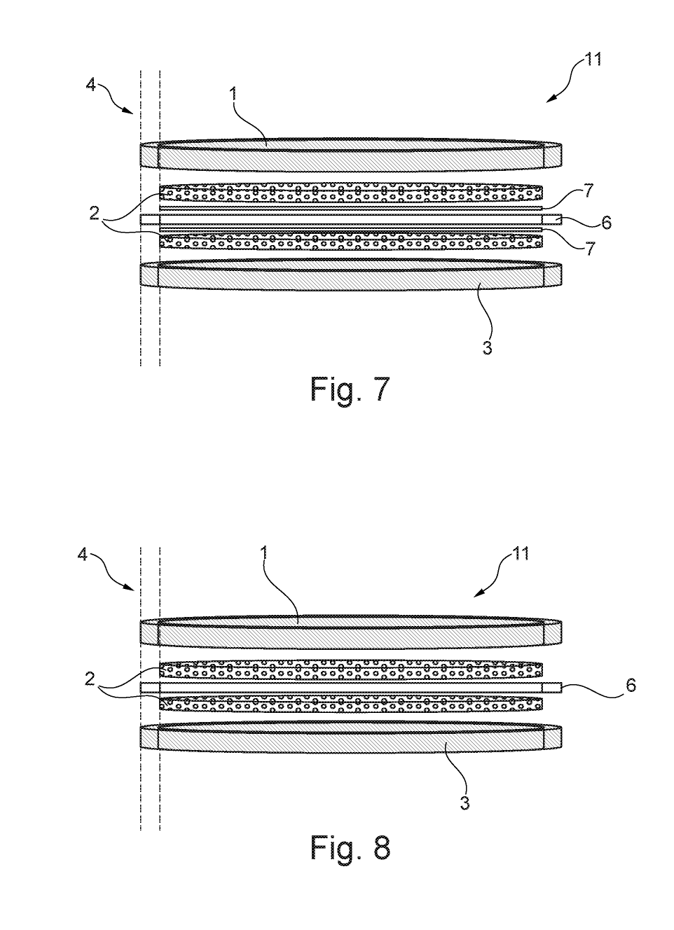

FIG. 7 shows a further embodiment example of a cosmetic pad 11. Individual layers of the cosmetic pad 11 are shown at a distance from each other to obtain a better overview.

The upper material of the cosmetic pad 11 has a fleece layer 1 and has the function of collecting and storing fluid. The fleece layer 1, also called absorber layer, borders on a layer of an absorbent core 2. This is followed by a gluing 7 and a fluid-impermeable layer 6, a further gluing 7, a further layer of the absorbent core 2 and a fleece layer 3.

To realise a better removal result a fluid-impermeable layer 6 separates two layers of the absorbent core 2 from each other in order to be able to ensure optimal double-sided use. The fluid-impermeable layer 6 can be a film in this embodiment example, but can also be a paper. The absorbent core 2 stores the fluid and serves for an appropriate dispensing of the fluid back into the upper/lower fleece layer 1, 3.

In this embodiment example the absorbent core 2 has only cellulose flakes. The absorbent core does not include a superabsorber.

As also described for FIG. 5 the fleece layer 1, 3 and the film 6 are firmly compressed under pressure in the edge area 4, so that a thin layer is created and the edge 4 forms a closed surface. The edge 4 ensures an adhesion of the various layers in order to maintain the particular absorption capacity inside the pad 11 and for avoiding a separation of the layers through rubbing on a surface. The edge 4 also additionally serves for realising an absolutely lint-free result.

The cosmetic pad has a diameter of approx. 5.5 cm and a thickness of approx. 3.5 to 5.0 mm.

FIG. 8 shows a further embodiment example of a cosmetic pad 11. Individual layers of the cosmetic pad 11 are shown at a distance from each other to obtain a better overview.

The construction of the cosmetic pad 11 is similar to the embodiment example from FIGS. 7 and 6, but without a gluing, so that a sequence of fleece layer 1, first part of the two-part absorbent core 2, fluid-impermeable layer 6, second part of the two-part absorbent core 2 and fleece layer 3 results. The description of FIG. 7 applies in context for FIG. 8.

As with the embodiment example from FIG. 7 the absorbent core 2 includes only cellulose flakes. The adhesion of the cellulose flakes to the fluid-impermeable layer 6 means that a gluing can be omitted.

The cosmetic pad has a diameter of approx. 5.5 cm and a thickness of approx. 3.5 to 5.5 mm.

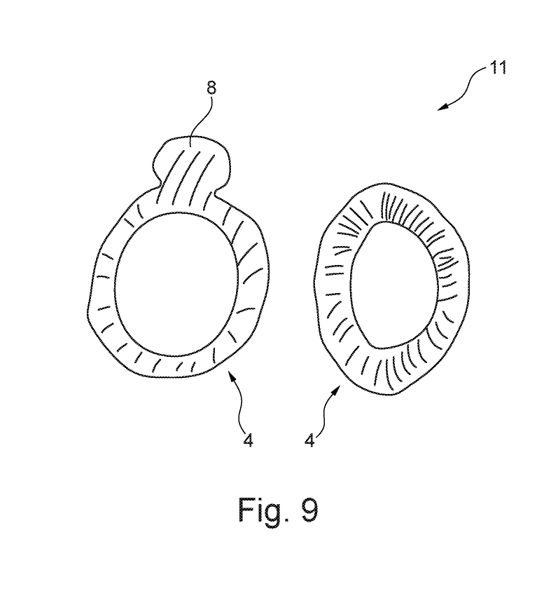

FIG. 9 shows two embodiment examples of an edge area 4 of a cosmetic pad. The edge area 4 is as described in the previous Figures, and is additionally compressed wide and/or corrugated. This enables particularly good access in the rounded corners or the edges of the nails or toe nails.

One of these embodiment examples with a wide embossed edge 4 has a tab 8 for a particularly easy removal of nail varnish from the nail edge.

It should be noted that the methods, devices and systems described in this document can be used on their own as well as in combination with other methods, devices and systems described in this document. All aspects of the methods, devices and systems described in this document can also be combined with each other in multiple ways. The characteristics of the claims can also be combined with each other in multiple ways.

The invention is described in detail with reference to the drawings and the above description. This invention can however be realised in many different forms and should not be interpreted as limited to the embodiments illustrated here; instead these embodiments are envisaged to ensure that this disclosure is thorough and complete, and cover the scope of protection of the invention completely for a person skilled in the art. The terminology used in the detailed description of the embodiments illustrated in the enclosed drawings is not intended to limit the invention. Identical symbols in the drawings refer to the same elements.

1. Cosmetic pad for removal of nail varnish remover or make-up remover, characterised in that it comprises an absorbent core (2) and a fleece layer (1).

2. Cosmetic pad according to claim 1, characterised in that it has a distribution covering (5) between the absorbent core and the fleece layer.

3. Cosmetic pad according to claim 1 or 2, characterised in that it has a fluid-impermeable layer (6), in particular a film or a paper, an absorbent core and a fleece layer.

4. Cosmetic pad according to one of the preceding claims, characterised in that it has a gluing (7) between the fleece layer and the distribution covering, or between the absorbent core and the fluid-impermeable layer.

5. Cosmetic pad according to claim 3 or 4, characterised in that a further absorbent core (2) and a further fleece layer (3) are also arranged on the exposed side of the fluid-impermeable layer.

6. Cosmetic pad according to one of the preceding claims, characterised in that the absorbent core and the various layers are connected in the edge area (4).

7. Cosmetic pad according to one of the preceding claims, characterised in that the pad is saturated with a fluid, in particular nail varnish remover or make-up remover.

8. Cosmetic pad according to one of the preceding claims, characterised in that the pad has a round, oval or rectangular shape.

9. Cosmetic pad according to one of the preceding claims, characterised in that the pad has a diameter, a length or a width of 2-10 cm, in particular of 3-6 cm, in particular of 5.5 cm or 4.5 cm.

10. Cosmetic pad according to one of the preceding claims, characterised in that the pad has a thickness of 3.0 mm to 6.0 mm, in particular of 3.5 mm to 5.0 mm, or of 3.0 mm to 4.5 mm.

11. Method for producing a cosmetic pad, in particular according to one of the claims 1 to 10.
